(19) United States
(12) Patent Application Publication
(10) Pub. No.: US 2025/0143933 A1
(43) Pub. Date: May 8, 2025

(54) ENDOSCOPIC, PERCUTANEOUS AND OTHER METHODS FOR DISSOLUTION OF PANCREATIC NECROSIS

(71) Applicants: The Johns Hopkins University, Baltimore, MD (US); University of Maryland, Baltimore, Baltimore, MD (US)

(72) Inventors: Venkata S. AKSHINTALA, Towson, MD (US); Stephen W. HOAG, Cantonsville, MD (US); Mouen A. KHASHAB, Towson, MD (US); Todd A. BRENNER, Baltimore, MD (US); Vikesh K. SINGH, Ellicott City, MD (US); Ayesha KAMAL, Baltimore, MD (US)

(21) Appl. No.: 18/684,329

(22) PCT Filed: Aug. 25, 2022
(86) PCT No.: PCT/US2022/041556

§ 371 Date: Feb. 16, 2024

Related U.S. Application Data
(60) Provisional application No. 63/236,966, filed on Aug. 25, 2021.

(51) Int. Cl. A61F 13/20; A61F 13/26; A61K 9/12; A61K 38/48; A61K 47/10; A61K 47/26; A61K 47/32; A61K 47/38; A61P 1/18

(52) U.S. Cl. CPC A61F 13/2074; A61F 13/26; A61K 9/122; A61K 38/4886; A61K 47/10; A61K 47/26; A61K 47/32; A61K 47/38; A61P 1/18; A61F 2013/2014

(57) ABSTRACT

An endoscopic foam delivery catheter comprising a dissolution solution and methods of their use for treating pancreatic necrosis are disclosed.

### BACKGROUND

Acute pancreatitis (AP) is the third most common gastrointestinal cause of hospitalization in the United States, resulting in more than 350,000 emergency room visits each year, with 70 percent of those patients requiring inpatient admission. Peery et al., 2019. Moreover, the incidence of AP is increasing worldwide due to increased rates of obesity and gallstone disease. The majority of AP cases are mild and self-limited, Yadav and Lowenfels, 2013. In 10 to 20 percent of cases, however, patients develop severe AP with necrosis of the pancreatic gland, resulting in the formation of a collection of necrotic material referred to as “walled-off pancreatic necrosis” (WON). Peery et al., 2019; Banks et al., 2013. This collection of necrotic material typically consists of a solid necrosome, which is a nidus for intra-abdominal and bloodstream infections, and a viscous liquid component containing pancreatic digestive enzymes (FIG. 1).

Patients with WON have prolonged, complex clinical courses with a mortality rate of up to 25 to 30 percent. Banks et al., 2013. The current standard of care for management of AP with WON is an endoscopic step-up technique in which a patient undergoes at least one, and often multiple, endoscopic procedures to create a tract between the stomach and the necrotic collection (FIG. 1A) to drain the liquid component of the collection, leaving the solid necrosome behind. Baron et al., 2020; van Brunschot et al., 2018.

In such procedures, at least one, and often several, stents are then placed to maintain the patency of this drainage tract, while additional drainage catheters are often inserted percutaneously or through the nose to allow for regular saline lavage and to optimize drainage. In recent years, larger diameter lumen apposing metal stents (LAMS), such as the AXIOS (Boston Scientific) and HOT SPAXUS (Taewoong Medical) systems, have increasingly been utilized in endoscopic transluminal drainage procedures to achieve more effective drainage (FIG. 2). It has been demonstrated previously that LAMS have a higher technical success rate in the treatment of WON (92% successful drainage) as compared to less expensive plastic stents (84% successful drainage). Chen et al., 2018. This observation has been validated elsewhere in the literature. Abu Dayyeh et al., 2018.

Patients who do not achieve adequate drainage and debridement of their collections through this approach may then undergo endoscopic necrosectomy (FIG. 1B) to remove the solid necrosome using a variety of endoscopic accessories such as snare, forceps, and sharp debridement tools. Gornals et al., 2016; Voermans et al., 2015; Siddiqui et al., 2017; Gardner et al., 2011. In one prospective randomized controlled trial of endoscopic step-up management, the MISER trial, 44% of patients with WON required more than one endoscopic procedure; 23% required a necrosectomy procedure as well, and an additional 8.8% required two or more necrosectomies. Bang et al., 2019. Despite this, 17.6% of patients still required additional percutaneous drainage catheter placement to facilitate drainage of the thick gluey necrotic debris. Bang et al., 2019. A similar prospective trial in Europe, the TENSION trial, showed that patients undergoing endoscopic step-up required a mean of three endoscopic procedures, including two necrosectomies, and 27% required percutaneous drainage in addition. van Brunschot et al., 2018. Patients on average required 14 hospital days between initial drainage and the first necrosectomy. van Brunschot et al., 2018.

As a result, the costs associated with both endoscopic re-intervention and prolonged hospital stays are marked. The mean cost of the index admission and follow-up care at six months for patients treated endoscopically in MISER was $75,830. Bang et al., 2019. Patients in the TENSION trial incurred costs of 60,228 euros for care in the first 6 months. van Brunschot et al., 2018. Several factors likely contribute to these costs, including long hospital stays necessitated by the slow drainage of necrotic debris, high costs associated with endoscopic procedures, and the need for expensive endoscopic devices LAMS and endoscopic grasper devices. The two marketed LAMS systems for instance cost $4,900 and $3,800, respectively, per unit. By comparison plastic stents cost roughly $104 on average. Chen et al., 2018.

Adding to these costs is the need for multiple interventions, often necessitated by stent clogging, inadequate drainage, migration of the LAMS, bleeding due to erosion into a vessel (FIG. 2), or viscus perforation. Fugazza et al., 2020. Each endoscopic LAMS placement costs on average $5,237, while each necrosectomy procedure costs an average of $3,786. Chen et al., 2018.

### SUMMARY

In some aspects, the presently disclosed subject matter provides an endoscopic foam delivery catheter comprising: a first syringe comprising a dissolution solution and a second syringe comprising a propellent, wherein the first syringe and the second syringe are adapted to be in fluid communication a Y-connector, wherein the Y-connector comprises a first intake port in fluid communication with the first syringe, and a second intake port in fluid communication with the second syringe, wherein the Y-connector is in fluid communication with a dual-lumen catheter via an output port, wherein the dual-lumen catheter comprises a propellent lumen and dissolution solution lumen, a dispersion screen; a dissolution foam outflow port at a distal end thereof.

In some aspects, the endoscopic foam delivery catheter further comprises a foam generation cap comprising a foam generation chamber, a dispersion screen, and a hollow cavity adapted for camera view. In some aspects, the foam generation cap further comprises a tool port plug. In some aspects, the foam generation cap further comprises an endoscope camera.

In some aspects of the endoscopic foam delivery catheter, the dissolution solution comprises between about 0.5% to about 5% hypochlorite and one or more of a gel formulation, a foam formulation, and combinations thereof.

In some aspects, the dissolution solution further comprises one or more dissolution compounds selected from chlorine dioxide, hydrogen peroxide, eusol (chlorinated lime in boric acid), ethylenediaminetetraacetic acid (EDTA), formocresol (formaldehyde, cresol, glycerin), glutaraldehyde, one or more proteolytic enzymes, peracetic acid, chlorhexidine, Ca hypochlorite, and papain gel. In some aspects, the one or more proteolytic enzymes include a collagenase.

In some aspects, the gel formulation comprises one or more components selected from an alginic acid, a cellulosic-based compound, a lectin-like cytoadhesive, gellan gum, polycarbophil, carbomer, chitosan, xanthan gum, cyclomethicone, a poloxamer, polyethylene oxide, carrageenan, carboxymethylcellulose, ammonium alginate, sodium alginate, potassium alginate, calcium alginate, poly (methyl vinyl ether/maleic anhydride) ethyl cellulose, propylene glycol, sodium hyaluronate, agar, dextran hydroxypropyl cellulose, croscarmellose sodium, dimethyl-beta-cyclodextrin, povidone, methylcellulose, potassium alginate, pectin, an aliphatic polyester, ceresin, a polyoxyethylene alkyl ester, ceratonia, gelatin, propylene glycol alginate, polyvinyl alcohol, hydroxyethyl methyl cellulose polyoxyl hydroxystearate, and guar gum.

In some aspects, the dissolution solution further comprises an excipient selected from glyceryl monooleate, lecithin, oleic acid, a dibutyl sebacate salt, sodium chloride, one or more preservatives, glycerin, chlorhexidine, dimethyl sulfoxide, glyceryl behenate, sodium stearate, glyceryl palmitostearate, olive oil, and sucrose stearate.

In some aspects, the foam formulation comprises one or more of a pharmaceutically acceptable vehicle, a surfactant, a propellant, a stabilizing polymer, and a buffer.

In some aspects, the pharmaceutically acceptable vehicle comprises propylene glycol and water.

In some aspects, the surfactant is a polysorbate or a sorbitan ester.

In some aspects, the propellant is selected from isobutane, propane, air, carbon dioxide, and combinations thereof.

In some aspects, the stabilizing polymer is a poloxamer.

In some aspects, the buffer is a phosphate or a citrate buffer.

In some aspects, the foam formulation comprises one or more components selected from a solvent, a foaming agent, a foam stabilizer, a hydrophobic component and/or emollient, an absorption promoter, a foam breaker, and an antifoaming agent.

In some aspects, the solvent is selected from distilled water, ethanol, isopropanol, glycerin, propylene glycol, dimethyl isosorbide, and dimethyl sulfoxide (DMSO).

In some aspects, the foaming agent is selected from cetyl alcohol, cetyl stearyl alcohol, sodium docecyl sulfate, sodium oleate, sodium stearate, stearic acid, and polysorbate 20.

In some aspects, the foam stabilizer is selected from xanthan gum, guar gum, hydroxyethyl cellulose, hydroxypropyl cellulose, hydroxypropyl methylcellulose methyl cellulose, agar-agar, alginates, sodium lauryl sulfate, lauryl acid, palmitic acid, stearic acid, coconut oil, tragacanth gum, gelatin, and glycerin.

In some aspects, the hydrophobic component and/or emollient is selected from a mineral oil, a plant oil, an ester, an essential oils, petrolatum, omega-3 polyunsaturated oil, and silicon oil.

In some aspects, the absorption promoter is selected from ethanol, a fatty acid, and a fatty alcohol.

In some aspects, the foam breaker is selected from an alkyl polysiloxane, an oil, an alcohol, a fatty alcohol, and acetone.

In some aspects, the antifoaming agent is selected from silicone oil, a glyceride, and polyamide.

In some aspects, the foam formulation comprises one or more components selected from NaClO, cetyl alcohol, a polysorbate, glycerin, a polyvinylpyrrolidone, a hydroxypropyl methylcellulose (HPMC), and water.

In some aspects, the foam formulation comprises between about 0.5% to about 5% NaClO, between about 0.1% to about 3.0% cetyl alcohol, between about 0.1% to about 2.5% of a polysorbate, between about 2.0% to about 6.0% glycerin, between about 0.0% to about 3.0% of a polyvinylpyrrolidone, between about 0.0% to about 3.0% of a hydroxypropyl methylcellulose (HPMC), and water.

In some aspects, the foam formulation comprises about 3.5% NaClO, about 0.5% cetyl alcohol, about 0.5% of a polysorbate, about 5.0% glycerin, between about 0.0% to about 3.0% of a polyvinylpyrrolidone, between about 0.0% to about 0.3% of a hydroxypropyl methylcellulose (HPMC), and water.

In other aspects, the presently disclosed subject matter provides a kit comprising the endoscopic foam delivery catheter as described hereinabove.

In some aspects, the kit further comprises one or more accessories for lavaging the one or more pancreatic necrotic collections.

In some aspects, the kit further comprises one or more accessories for draining the one or more pancreatic necrotic collections.

In other aspects, the presently disclosed subject matter provides a method for treating pancreatic necrosis, the method comprising administering a dissolution solution with the endoscopic foam delivery catheter as described hereinabove to one or more pancreatic necrotic collections in a subject in need of treatment thereof.

In some aspects, the method comprises dissolving one or more solid necrosomes of the one or more pancreatic necrotic collections.

In some aspects, the method further comprises decreasing a viscosity of a liquid collection of the one or more pancreatic necrotic collections.

In some aspects, the method further comprises lavaging the one or more pancreatic necrotic collections.

In some aspects, the method further comprises draining the one or more pancreatic necrotic collections with a stent or a pigtail drain.

In some aspects, a volume of the dissolution solution administered has a range from about 10 mL to about 100 mL.

In some aspects, the method comprises administering the dissolution solution over a period of time having a range from about 1 minute to about 10 minutes.

In some aspects, the treatment is conducted over one treatment session.

In other aspects, the presently disclosed subject matter provides a dissolution solution as described hereinabove.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Drawings, which are not necessarily drawn to scale, and wherein:

FIG. 1A and FIG. 1B show endoscopic transluminal drainage of liquid component (FIG. 1A) and endoscopic necrosectomy of solid necrosome component (FIG. 1B) (images from Pancreapedia);

FIG. 2 shows placement of lumen apposing metal stent (LAMS) to connect the gastriclumen and the pancreatic necrotic collection (Image 1); the necrotic collection after necrosectomy (Image 2); and bleeding due to LAMS/necrosectomy (Image 3) (images from Pancreapedia);

FIG. 3 shows preliminary results of near infrared spectroscopy of human pancreas necrosis debris showing the presence of complex protein and lipids;

FIG. 4 is a petri dish containing dilute sodium hypochlorite 2.5% and human pancreas necrosis debris. The debris breaks down within minutes in the presence of the sodium hypochlorite;

FIG. 5. is a schema of endoscopic delivery of pancreas necrosis dissolution chemicals during cyst gastrostomy. Modified from www.pancreapedia.org/reviews/endoscopic-treatment-of-infected-necrosis;

FIG. 6 is a schematic of an endoscopic/percutaneous foam delivery catheter comprised of a dual-lumen catheter with a distal foam proportioning system;

FIG. 7 is an enlarged schematic of an endoscopic/percutaneous foam delivery catheter detailing the foam proportioning system;

FIG. 8 is a schematic showing a representative topical foam application process;

FIG. 9 is a depiction of representative pancreas necrosis dissolution experiments;

FIG. 10 is a representative foam generation endoscopic cap;

FIG. 11A, FIG. 11B, FIG. 11C, FIG. 11D, and FIG. 11E show different views of a foam generator for endoscopy. FIG. 11A is a conventional endoscopic cap known in the art; FIG. 11B is an endoscope distal tip comprising a foam generation endoscopic cap; FIG. 11C is a section view of a foam generation endoscopic cap comprising a foam generation chamber, a dispersion screen, and a camera view cavity; FIG. 11D is a bottom section view of a foam generation endoscopic cap illustrating flow of the presently disclosed solution and air/CO₂ into the cap and foam out of the cap; and FIG. 11E is a side section view of a foam generation endoscopic cap showing a tool port plug and an endoscope camera; and

FIG. 12A and FIG. 12B are photographs showing a prototype foam generation endoscopic cap. FIG. 12A is a photograph of two syringes adapted for providing the presently disclosed solution and air/CO₂ to the cap; and FIG. 12B is a photograph showing a prototype foam generation endoscopic cap with a custom-sealed fitting for tubing and generation of a low-stiffness foam.

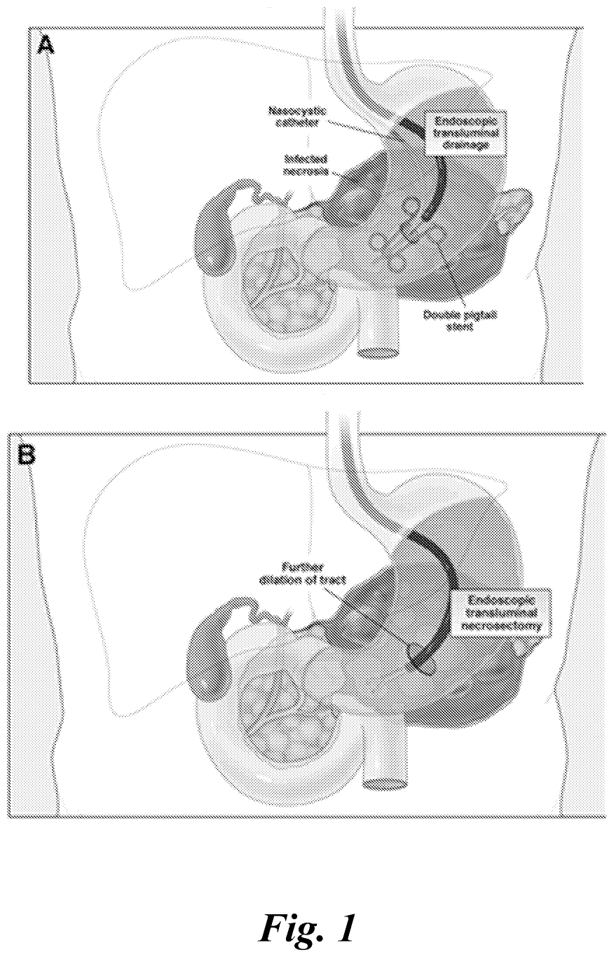

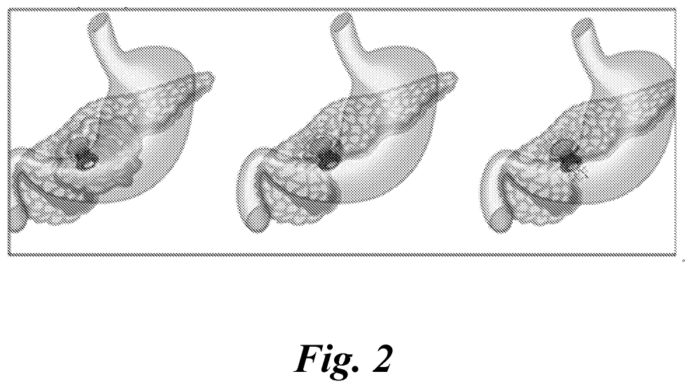

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Endoscopic, Percutaneous and Other Methods for Dissolution of Pancreatic Necrosis

As provided hereinabove, acute pancreatitis (AP) is among the most common gastrointestinal illnesses. Around 10 to 20% of patients with AP develop walled-off necrosis (WON) of the pancreatic gland, peripancreatic tissue, or both. This necrosome contains both solid and viscous liquid components, which can become a nidus for infection and are difficult to drain. The standard management of AP with WON involves drainage and debridement of the necrotic collection using endoscopic, percutaneous, and surgical interventions to adequately drain and debride the collection. Multiple surgical interventions can be required, adding to the cost and morbidity and mortality rates.

An objective of the presently disclosed subject matter is to develop a chemical debridement formulation designed to dissolve the solid necrosome and decrease the viscosity of the liquid collection to facilitate rapid and complete drainage of WON in a single session. More particularly, the presently disclosed subject matter provides delivery of a chemical dissolution agent directly to the necrosome to facilitate the dissolution of the WON and allow for more effective drainage through standard plastic stents and pigtail drains, without the need for expensive endoscopic interventions and associated accessories, such as lumen apposing metal stents and mechanical debridement tools.

Previous attempts to develop chemical dissolution agents to aid in endoscopic necrosectomy, including hydrogen peroxide (H₂O₂), tissue plasminogen activator, streptokinase, collagenases, and other proteases, have had minimal success. Cobankara et al., 2010; Stojicic et al., 2010; Gupta et al., 2014; Bhargava et al., 2021; Parra et al., 2015; Günay et al., 2021; Maharshi et al., 2021; Messallam et al., 2021; Abdelhafez et al., 2013; Othman et al., 2017.

In contrast, in some embodiments, the presently disclosed subject matter provides a sodium hypochlorite-based dissolution composition designed to be delivered via endoscope and/or percutaneous catheter. In some embodiments, sodium hypochlorite-based dissolution composition is administered in combination with additional candidate dissolution compounds including, but not limited to, chlorine dioxide, hydrogen peroxide, eusol (chlorinated lime in boric acid), ethylenediaminetetraacetic acid (EDTA), formocresol (formaldehyde, cresol, glycerin), glutaraldehyde, collagenase, proteolytic enzyme combinations, peracetic acid, chlorhexidine, Ca hypochlorite, and papain gel.

The presently disclosed subject matter draws on the extensive experience and literature of the oral-maxillofacial and dental surgery fields, which have long applied chemical debridement techniques for difficult-to-access periodontal abscesses and root-canal preparation. Sodium hypochlorite (NaOCl) is the most commonly used endodontic irrigant worldwide due to its antimicrobial and tissue-dissolving activity and has been extensively studied. Cobankara et al., 2010; Stojicic et al., 2010; de Almeida et al., 2015; Senia et al., 1971; Shih et al., 1970; Taylor et al., 1918; Svec and Harrison, 1977; Cunningham and Balekjian, 1980; Cullen et al., 2015; Thé et al., 1980. Further, stabilized sodium hypochlorite solution has been used in the treatment of diffuse peritonitis. Sukovatykh et al., 2008; Sukovatykh et al., 2009; Sukovatykh et al., 2011; Sukovatykh et al., 2012. The treatment of diffuse peritonitis, however, is an anti-inflammatory use, which is distinct from presently disclosed dissolution approach for the removal of WON.

Sodium hypochlorite reacts with water to produce hypochlorous acid (HOCl⁻) and hypochlorite ions (OCl⁻), which cause fat degradation, saponification, and amino acid degradation. Moorer and Wesselink, 1982. Without wishing to be bound to any one particular theory, it is thought that, by using a sodium hypochlorite-based dissolution compound to dissolve and liquify the solid necrosome to facilitate more rapid and complete drainage of WON collections, the need for repeat endoscopic procedures, expensive endoscopic accessories, and the extensive costs and complications associated with these interventions will be minimized.

Representative dissolution agents are provided in Table 1. These agents, and any agents described herein, can be administered simultaneously or sequentially, e.g., simultaneously in a single formulation or sequentially in individual formulations or sequentially with one or more agents combined in one or more formulations. When administered sequentially, the sequence can vary depending on the application and the individual agents administered.

TABLE 1
Representative Dissolution Agents

| | Typical | Mechanism | |
|---|---|---|---|
| | Concentration | of Action | |
| Agent | Range | (MOA) | Effects/Uses |
| NaOCl | 1%, 2%, 4%, | NaOCl reacts | Dissolution of |
| | 5.25%, 5.8%, | with water to | tissue increased |
| | 6%, 8.25% | produce | linearly with the |
| | | hypochlorous | increasing |
| | | acid (HOCl⁻) | concentration of |
| | | and | NaOCl. |
| | | hypochlorite | It has been shown |
| | | ions (OCl⁻) | that the use of |
| | | which cause fat | 5.25% doesn't |
| | | degradation, | result in |
| | | saponification, | increased clinical |
| | | and amino acid | toxicity. |
| | | degradation. | EDTA and |
| | | | dentine |
| | | | negatively affect |
| | | | the dissolution |
| | | | capacity of |
| | | | NaOCl. |
| | | | Effect of dentin is |
| | | | minimized by |
| | | | frequently |
| | | | refreshing the |
| | | | solution. |
| | | | Efficacy of |
| | | | 8.25% solution |
| | | | has been seen, |
| | | | but clinical |
| | | | toxicity profiles |
| | | | are lacking. |
| Chlorine dioxide | 13.8% | It's a strong | The effect has been |
| | | oxidizing agent, | shown to be similar |
| | | which breaks | to 5.8% NaOCl. |
| | | molecular | |
| | | bonds of the | |
| | | cell membrane | |
| | | resulting in the | |
| | | death of the | |
| | | organism. | |
| Hydrogen peroxide | 3%, 30% | Breaks DNA | Antimicrobial |
| | | double-strand | action is seen at |
| | | by oxidization | lower |
| | | through reactive | concentrations |
| | | oxygen species. | also. |
| | | | Higher |
| | | | concentration has |
| | | | a sporicidal |
| | | | effect. |
| | | | Concentrations |
| | | | higher than 30% |
| | | | percent have been |
| | | | shown to be toxic |
| | | | causing local |
| | | | damage. |
| Eusol (Chlorinated lime | 73.3% | Acts by | Used for wound |
| in boric acid) | | releasing | disinfection, ulcer |
| Contains 0.25% | | nascent chlorine | cleaning, and wet |
| weight/volume of | | which acts as a | dressing. |
| available chlorine | | desloughing | It has antiseptic |
| with a pH between | | agent. | properties and can also |
| 7.5 and 8.5. | | | be used as a normal |
| | | | disinfectant. |
| Formocresol | 1.5% solution | The | Originally, the |
| (formaldehyde, | (in dentistry | formaldehyde | aim of using |
| cresol, | no standard | component of | formocresol was |
| glycerine) | concentration | formocresol | to completely |
| | and it varies | is strongly | mummify (fix) all |
| | widely from | bactericidal | residual pulpal |
| | 1% to 10%) | and reversibly | tissue and |
| | | inhibits many | necrotic material |
| | | enzymes in the | within the root |
| | | inflammatory | canal. |
| | | process. | Formaldehyde |
| | | | components |
| | | | remain a |
| | | | genotoxic and |
| | | | carcinogenic |
| | | | problem |
| | | | worldwide, hence |
| | | | utilization has |
| | | | decreased in |
| | | | dentistry. |
| Glutaraldehyde | | | |
| Collagenase | | A proteolytic | Used for wound |
| | | enzyme that | debridement |
| | | has a high | in clinical |
| | | specificity for | practice in |
| | | native and | patients with |
| | | denatured | burns, pressure, |
| | | collagen in | and venous |
| | | necrotic tissue. | ulcers. |
| Ethylenediaminetetraacetic | | Organizes the | |
| acid (EDTA) | | debris for | |
| | | subsequent | |
| | | dissolution | |
| Other proteolytic enzymes | | | |
| Peracetic acid | 5% | | |
| Chlorhexidine | 2% | | |
| Ca hypochlorite | 5%, 10% | | |
| Papain gel | 8% | | |

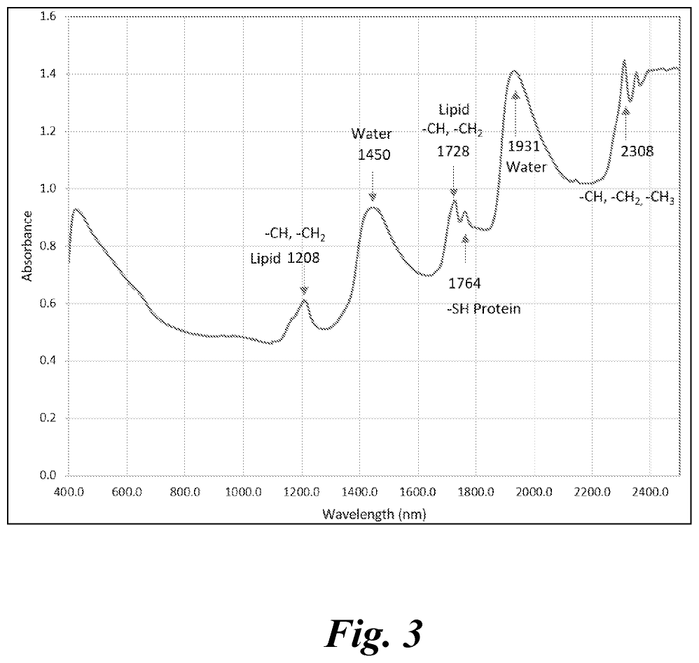

To perform preliminary proof-of-concept testing, solid WON debris was collected from seven patients undergoing endoscopic necrosectomy. The chemical composition of the debris was characterized using near-infrared spectroscopy analysis, which demonstrated the presence of both lipids and proteins (FIG. 3). Several candidate compounds that might be suitable, alone or in combination, for WON debris dissolution were identified. The candidate debridement compounds were tested on ex vivo samples to assess their capacity for dissolving WON debris. Interestingly, lipophilic solvents, such as acetone, which tend to combine with or dissolve in lipids or fats, had minimal to no effect on WON debris, suggesting the predominant presence of protein or complex lipids.

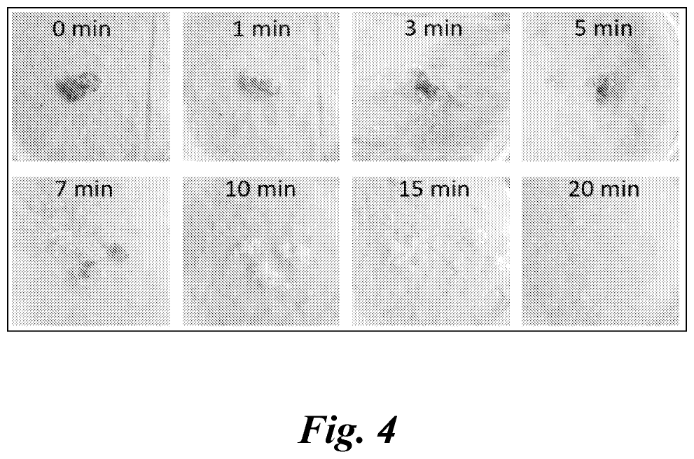

The most promising dissolution compound in this initial screen was NaOCl, which demonstrated excellent solvent properties in the WON samples even at a modest concentration of 5% (FIG. 4). These results are similar to dental surgery studies that have found rapid pulp dissolution at 8.25% NaOCl. Cullen et al., 2015.

In further embodiments, the dissolution compounds of interest can be combined with gel polymers, which are pharmacologically inert, biocompatible and biodegradable and include, but are not limited to alginic acid/alginates, cellulosic-based compounds, lectin-like cytoadhesives and gellan gum, polycarbophil, carbomer, chitosan, sodium alginate, xanthan gum, cyclomethicone, poloxamer, polyethylene oxide, carrageenan, carboxymethylcellulose, alginates (ammonium, sodium, potassium, calcium), poly(methyl vinyl ether/maleic anhydride) ethyl cellulose, propylene glycol, sodium hyaluronate, agar, dextran hydroxypropyl cellulose, croscarmellose sodium, dimethyl-beta-cyclodextrin, povidone, methylcellulose, potassium alginate, pectin, aliphatic polyesters, ceresin, polyoxyethylene alkyl esters, ceratonia, gelatin, propylene glycol alginate, polyvinyl alcohol, hydroxyethyl methyl cellulose polyoxyl hydroxystearate, guar gum and helper excipients such as, but not limited to glyceryl monooleate, lecithin, oleic acid, dibutyl sebacate salts, such as sodium chloride, preservatives, glycerin, chlorhexidine, dimethyl sulfoxide, glyceryl behenate, sodium stearate, glyceryl palmitostearate, olive oil, and sucrose stearate.

To minimize the procedure duration and residence time, the combination of dissolution chemicals and gel polymer will be optimized to percolate throughout the necrotic cavity rapidly. In some embodiments, the presently disclosed dissolution composition comprises a foam formulation that will easily disperse around the necrotic tissue facilitating tissue breakdown. The foam can have a low viscosity for easy dispersion, combined with viscoelastic solid characteristics, so it stays in place during the procedure, to reduce off target side effects.

Foams are colloidal systems in which a gas is dispersed in a liquid. Techniques for producing foam sprays are well-characterized in the pharmaceutical and cosmetic literature. A representative formulation is provided in Table 2.

TABLE 2
Representative foam formulation

| Ingredient | Amount |
|---|---|
| API(s) | 1 part |
| Vehicle | 2 parts (Water & propylene glycol) |
| Surfactant | 5% (Tween or Span) |
| Propellant | 1 part isobutane and propane |
| Stabilizing polymer | 4% (poloxamer 188) |
| Buffer | 2% (phosphate or citrate) |

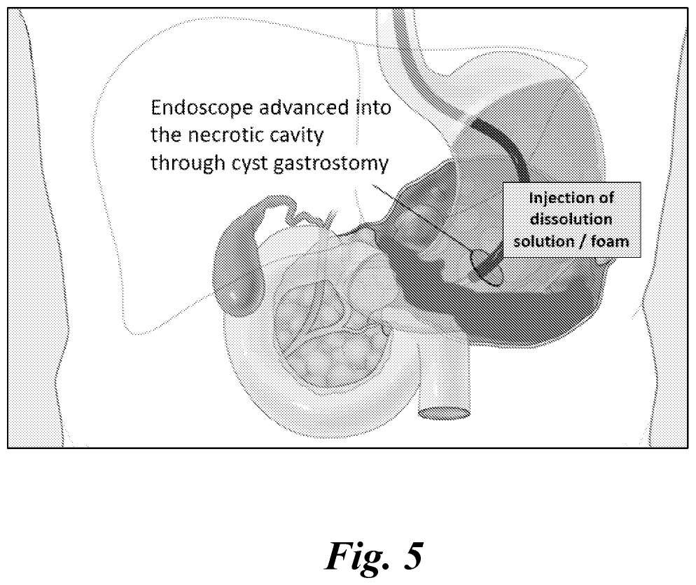

This formulation will be tested for foaming properties. A minimally acceptable formulation will be validated ex vivo to quantify the degree of chemical WON dissolution and assess the minimal orifice through which the resulting fluid can be drained. The final dissolution foam formulation will be delivered via a standard endoscope and injected into the necrotic collection using a standard injection needle or by catheter delivery through the tract created between stomach and the necrotic collection (FIG. 5).

In other embodiments, the presently disclosed subject matter provides an integrated foam delivery catheter system for seamless delivery of the foam via endoscopic needle, endoscopic catheter, or percutaneous catheter. The foam's physical properties, including foam expansion ratio, stability, viscosity, viscoelastic tan δ and stability, can be optimized for delivery through the small orifices of standard endoscopic and/or percutaneous transcatheter delivery modalities. Procedural protocols, including dissolution foam delivery volumes, rates, and dwell times, will also be developed and validated for optimal necrosome dissolution.

Accordingly, in some embodiments, the presently disclosed subject matter provides: (1) use of candidate chemicals for chemical dissolution of WON; (2) specific delivery methods using a foam-based multiphase system; (3) foam compositions that are compatible with dissolution chemicals; (4) combinations of fixative agents with the dissolution chemicals to improve efficacy; and (5) use of a foam delivery kit compatible with an endoscope system.

More particularly, in some embodiments as provided herein below, the presently disclosed subject matter provides an endoscopic foam delivery catheter comprising: a first syringe comprising a dissolution solution and a second syringe comprising a propellent, wherein the first syringe and the second syringe are adapted to be in fluid communication a Y-connector, wherein the Y-connector comprises a first intake port in fluid communication with the first syringe, and a second intake port in fluid communication with the second syringe, wherein the Y-connector is in fluid communication with a dual-lumen catheter via an output port, wherein the dual-lumen catheter comprises a propellent lumen and dissolution solution lumen, a dispersion screen; a dissolution foam outflow port at a distal end thereof.

In some embodiments as provided herein below, the endoscopic foam delivery catheter further comprises a foam generation cap comprising a foam generation chamber, a dispersion screen, and a hollow cavity adapted for camera view. In some aspects, the foam generation cap further comprises a tool port plug. In some aspects, the foam generation cap further comprises an endoscope camera.

In some embodiments, the presently disclosed subject matter provides a composition comprising 0.5% to about 5% hypochlorite and one or more of a gel formulation, a foam formulation, and combinations thereof. In certain embodiments, the composition further comprises one or more dissolution compounds selected from chlorine dioxide, eusol (chlorinated lime in boric acid), ethylenediaminetetraacetic acid (EDTA), formocresol (formaldehyde, cresol, glycerin), hydrogen peroxide, glutaraldehyde, collagenase, one or more proteolytic enzymes, peracetic acid, chlorhexidine, Ca hypochlorite, and papain gel. In particular embodiments, the one or more proteolytic enzymes include a collagenase.

In certain embodiments, the composition comprises a gel formulation. In particular embodiments, the gel formulation comprises one or more components selected from an alginic acid, a cellulosic-based compound, a lectin-like cytoadhesive, gellan gum, polycarbophil, carbomer, chitosan, xanthan gum, cyclomethicone, a poloxamer, polyethylene oxide, carrageenan, carboxymethylcellulose, ammonium alginate, sodium alginate, potassium alginate, calcium alginate, poly(methyl vinyl ether/maleic anhydride) ethyl cellulose, propylene glycol, sodium hyaluronate, agar, dextran hydroxypropyl cellulose, croscarmellose sodium, dimethyl-beta-cyclodextrin, povidone, methylcellulose, potassium alginate, pectin, an aliphatic polyester, ceresin, a polyoxyethylene alkyl ester, ceratonia, gelatin, propylene glycol alginate, polyvinyl alcohol, hydroxyethyl methyl cellulose polyoxyl hydroxystearate, and guar gum.

In certain embodiments, the composition comprises an excipient selected from glyceryl monooleate, lecithin, oleic acid, a dibutyl sebacate salt, sodium chloride, one or more preservatives, glycerin, chlorhexidine, dimethyl sulfoxide, glyceryl behenate, sodium stearate, glyceryl palmitostearate, olive oil, and sucrose stearate.

In certain embodiments, the composition comprises a foam formulation. In particular aspects, the foam formulation comprises one or more of a pharmaceutically acceptable vehicle, a surfactant, a propellant, a stabilizing polymer, and a buffer.

In particular embodiments, the pharmaceutically acceptable vehicle comprises propylene glycol and water. In particular aspects, the surfactant is a polysorbate or a sorbitan ester. In particular aspects, the propellant is selected from isobutane, propane, and combinations thereof. In particular aspects, the stabilizing polymer is a poloxamer. In particular aspects, the buffer is a phosphate or a citrate buffer.

In other embodiments, the presently disclosed foam formulation comprises one or more components selected from a solvent, a foaming agent, a foam stabilizer, a hydrophobic component and/or emollient, an absorption promoter, a foam breaker, and an antifoaming agent.

In certain embodiments, the solvent is selected from distilled water, ethanol, isopropanol, glycerin, propylene glycol, dimethyl isosorbide, and DMSO.

In certain embodiments, the foaming agent is selected from cetyl alcohol, cetyl stearyl alcohol, sodium docecyl sulfate, sodium oleate, sodium stearate, stearic acid, and polysorbate 20.

In certain embodiments, the foam stabilizer is selected from xanthan gum, guar gum, hydroxyethyl cellulose, hydroxypropyl cellulose, hydroxypropyl methylcellulose methyl cellulose, agar-agar, alginates, sodium lauryl sulfate, lauryl acid, palmitic acid, stearic acid, coconut oil, tragacanth gum, gelatin, and glycerin.

In certain embodiments, the hydrophobic component and/or emollient is selected from a mineral oil, a plant oil, an ester (e.g., isopropyl myristate), an essential oils, petrolatum, omega-3 polyunsaturated oil, and silicon oil.

In certain embodiments, the absorption promoter is selected from ethanol, a fatty acid, and a fatty alcohol.

In certain embodiments, the foam breaker is selected from an alkyl polysiloxane, an oil, an alcohol, a fatty alcohol, and acetone.

In certain embodiments, the antifoaming agent is selected from silicone oil, a glyceride, and polyamide.

In particular embodiments, the foam solution comprises between about 0.5% to about 5% NaClO, including about 0.5%, 0.6%, 0.7%, 0.8%, 0.9%, 1.0%, 2.0%, 3.0%, 4.0%, and 5% NaClO, between about 0.1% to about 3.0% cetyl alcohol, including about 0.1%, 0.2%, 0.3%, 0.4%, 0.5%, 1.0%, 2.0%, and 3.0% cetyl alcohol, between about 0.1% to about 2.5% of a polysorbate, including about 0.1%, 0.2%, 0.3%, 0.4%, 0.5%, 1.0%, 1.5%, 2.0%, and 2.5% of a polysorbate, between about 2.0% to about 6.0% glycerin, including about 2.0%, 2.5%, 3.0%, 3.5%, 4.0%, 4.5%, 5.0%, 5.5%, and 6.0% glycerin, between about 0.0% to about 3.0% of a polyvinylpyrrolidone, including about 0.0%, 0.1%, 0.2%, 0.3%, 0.4%, 0.5%, 1.0%, 1.5%, 2.0%, 2.5%, and 3.0% of a polyvinylpyrrolidone, between about 0.0% to about 3.0% of a hydroxypropyl methylcellulose (HPMC), including about 0.0%, 0.1%, 0.2%, 0.3%, 0.4%, 0.5%, 1.0%, 1.5%, 2.0%, 2.5%, and 3.0%, and q.s. water, e.g., as much water as is sufficient for the formulation.

Polysorbates include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate). In particular embodiments, the polysorbate is tween™ 80, also referred to as polysorbate 80.

Polyvinylpyrrolidone (PVP), also commonly called polyvidone or povidone, is a water-soluble polymer made from the monomer N-vinylpyrrolidone and is available in a range of molecular weights and related viscosities. The following table provides representative PVP grades and their approximate molecular weight in Daltons.

TABLE 3
Representative PVP Grades

| | Molecular Weight |
|---|---|
| PVP | (Daltons) |
| PVP K-12 | 2500 |
| PVP K-15 | 8000 |
| PVP K-17 | 10,000 |
| PVP K-25 | 30,000 |
| PVP K-30 | 50,000 |
| PVP K-60 | 4,00,000 |
| PVP K-90 | 10,00,000 |
| PVP K-120 | 30,00,000 |

In particular embodiments, the polyvinylpyrrolidone is providone K-90.

Representative hydroxypropyl methylcelluloses include those in the METHOCEL™ family. The METHOCEL™ product range encompasses methylcellulose and hydroxypropyl methylcellulose (hypromellose), each available in different grades to address a particular application. For example, the following METHOCEL™ products are available (with the viscosity grade in mPa): E3 LV (3), E5 LV (5), E6 LV (6), E15 LV (15), E50 LV (50), E4M (4,000), E10M (10,000), VLV (2.8), F4 LV (4), F5 LV (5), F4M (4,000), K3 LV (3), K100 LV (100), K4M (4,000), K15M (15,000), K100M (100,000), K200M (200,000), A15 LV (15), and A4M (4,000). In particular embodiments, the hydroxypropyl methylcellulose (HPMC) is METHOCEL™ E4M.

In particular embodiments, the foam formulation comprises about 3.5% NaClO, about 0.5% cetyl alcohol, about 0.5% of a polysorbate, about 5.0% glycerin, between about 0.0% to about 3.0% of a polyvinylpyrrolidone, between about 0.0% to about 0.3% of a hydroxypropyl methylcellulose (HPMC), and q.s. water.

In other embodiments, the presently disclosed subject matter provides a method for treating pancreatic necrosis, the method comprising administering a presently disclosed composition to one or more pancreatic necrotic collections in a subject in need of treatment thereof.

In certain embodiments, the composition is administered to the one or more pancreatic necrotic collections by endoscopic delivery, percutaneous catheter, or a combination thereof. In particular embodiments, the composition is administered with the presently disclosed endoscopic foam delivery catheter.

In certain embodiments, the method comprises dissolving one or more solid necrosomes of the one or more pancreatic necrotic collections. In certain embodiments, the method comprises decreasing a viscosity of a liquid collection of the one or more pancreatic necrotic collections. In certain embodiments, the method further comprises lavaging the one or more pancreatic necrotic collections. In certain embodiments, the method further comprises draining the one or more pancreatic necrotic collections with a stent or a pigtail drain. In certain embodiments, a volume of the composition administered has a range from about 10 mL to about 100 mL. In certain embodiments, administering the composition occurs over a period of time having a range from about 1 minute to about 10 minutes. In certain embodiments, the treatment is conducted over one treatment session.

In other embodiments, the presently disclosed subject matter provides a kit comprising the presently disclosed endoscopic foam delivery catheter and/or the foam generation cap. In certain embodiments, the kit further comprises one or more accessories for lavaging the one or more pancreatic necrotic collections. In certain embodiments, the kit further comprises one or more accessories for draining the one or more pancreatic necrotic collections.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly the presently disclosed dissolution compositions and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the dissolution compositions described herein can be administered alone or in combination with adjuvants that enhance stability of the compositions, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the presently disclosed compositions and at least one additional therapeutic agent can be varied so long as the beneficial effects of the combination of these agents are achieved. Accordingly, the phrase “in combination with” refers to the administration of the presently disclosed compositions and at least one additional therapeutic agent either simultaneously, sequentially, or a combination thereof. Therefore, a subject administered a combination of the presently disclosed compositions and at least one additional therapeutic agent can receive the presently disclosed compositions and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed compositions and at least one additional therapeutic agent are administered simultaneously, they can be administered to the subject as separate pharmaceutical compositions, each comprising either the presently disclosed compositions or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of the presently disclosed composition and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Representative Foam Formulations 1-6

In form formulations 1-6, foams were made according to the methods described herein below. These foams were then evaluated in vitro and ex vivo.

For these examples, the foam solution was prepared with cetyl alcohol, tween 80, glycerin, providone k90, METHOCEL™ E4M and distilled water. The cetyl alcohol, tween 80, glycerin, providone k90 were heated and stirred at 70° C. until completely dissolved to form the mixture; then METHOCEL™ E4M was added. Distilled water (70° C.) was slowly incorporated into the mixture with continuous agitation (ultrasonic) until attending the final total volume.

TABLE 4
Foam Formulations 1 to 6

| Ingredient | Example 1 | Example 2 | Example 3 | Example 4 | Example 5 | Example 6 | Range |
|---|---|---|---|---|---|---|---|
| NaOCl | 3.5% | 3.5% | 3.5% | 3.5% | 3.5% | 3.5% | 0.5- |
| | | | | | | | 5% |
| Tween 80 | 0.5% | 0.5% | 0.5% | 0.5% | 0.5% | 0.5% | 0.1- |
| | | | | | | | 2.5% |
| METHOCEL ™ | 0.3% | 0.5% | 0.0% | 0.3% | 0.3% | 0.3% | 0.25- |
| E4M | | | | | | | 3.0%† |
| PVP k90 | 0.0% | 0.0% | 1.0% | 0.0% | 0.0% | 1.0% | 0.5 to |
| | | | | | | | 3.0%† |
| Glycerin | 5.0% | 5.0% | 5.0% | 0.0% | 5.0% | 5.0% | 2.0- |
| | | | | | | | 6% |
| Cetyl | 0.5% | 0.5% | 0.5% | 0.0% | 0.0% | 0.5% | 0.1- |
| Alcohol | | | | | | | 3.0 |
| Water | qs‡ | qs | qs | qs | qs | qs | qs |

‡qs is the Latin abbreviation for add sufficient quantity, in this case water.
†other grades of METHOCEL ™ and PVP can be used and would have different concentration ranges. Thus, the presently disclosed subject matter contemplates and encompasses all equivalent grades and their equivalent concentrations.

The following formulations formed foams, the quality varied with the amount of excipients.

### Example 2

### Foam Stability Studies

The stability of form formulations 1-6 was measured by the following equation:

$$foam height / liquid height = hf / hl$$

according to the following schematic:

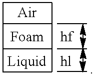

For this ratio, the bigger the number, the more foam is formed. For a given foam production there is an optimal ratio. In these studies, the initial foam ratio was measured right after foam formation (t0). The ratio was then measured without additional processing at 18 hrs (t18). If the number decreased over time this means the foam phase decreased and the liquid increased, which is an indication of foam stability. Here again there is an optimal foam stability. Results from the foam stability studies showing foam stability are provided in Table 5.

TABLE 5
Foam/solution (volume, %)

| | |
|---|---|
| 100% | 2*dilution |

| | t0 | 18 hrs | t0 | 18 hrs |
|---|---|---|---|---|
| Example 1 | 125 | 112.5 | 125 | 125 |
| Example 2 | 75 | 62.5 | 100 | 87.5 |
| Example 3 | 125 | 112.5 | 100 | 100 |
| Example 4 | 150 | 37.5 | 175 | 100 |
| Example 5 | 150 | 37.5 | 175 | 100 |
| Example 6 | 100 | 100 | 125 | 125 |

### Example 3

### Endoscopic, Percutaneous and Other Methods for Dissolution of Pancreatic Necrosis

### 3.1 Ex Vivo Evaluation

Further ex vivo testing will allow for the identification of an optimal solvent combination and concentration for the dissolution of WON using a larger sample size of WON debris. Additional WON debris samples will be collected from patients undergoing endoscopic necrosectomy, as successfully done in the past, using institutional review board (IRB) protocols already in place at the Johns Hopkins Hospital. Automated, high-throughput methods will be used for ex-vivo dissolution studies using NaOCl and other candidate agents (listed hereinabove) at various concentrations, for example, about 0.5% to about 5%, including about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0, or in combinations.

Scanning electron microscopy (SEM) will be used to perform tissue structure analysis. Chemical methods (for example, gas chromatography, colorimetry-based lipid and protein quantification kits) to identify the changes to WON debris on exposure to the candidate chemicals.

To assess WON breakdown in terms of clinical relevance, the minimum orifice diameter that can effectively drain the debris after dissolution, mimicking endoscopy or percutaneous suction, will be identified.

One goal in ex vivo testing will be to identify a chemical or combination of chemical agents that will result in greater than about 80% dissolution of WON debris at the lowest concentrations.

A second goal in ex vivo testing will be to conduct high-throughput safety studies using porcine granulation tissue and major blood vessels as a model system. At present, no reliable large animal model exists for WON. Therefore, preliminary ex-vivo safety studies of target dissolution chemicals will be conducted on porcine abscess wall granulation tissue, which will mimic the wall of a WON and adjacent large mesenteric blood vessels. Porcine tissue samples will be generated using previously described protocols in which subcutaneous abscesses are generated by subcutaneously injecting trinitrobenzenesulfonic acid in adult pigs. Wang et al., 2020.

The abscess wall will be harvested and a histopathological examination will be done to examine the integrity of granulation tissue and blood vessel walls after exposure to the dissolution chemicals at different residence times. These studies will seek to identify dissolution formulations resulting in no significant changes (less than about 10%) to blood vessel or granulation tissue integrity at the longest residence times.

A third goal in ex vivo testing will be the adaptation of the target dissolution chemical(s) to a foam-based formulation as described hereinabove.

### 3.2 In Vivo Testing

Once a chemical dissolution formulation is validated by ex vivo testing and adapted to a foam-based delivery platform, in vivo testing will be conducted using the porcine subcutaneous abscess model described above. As above, the goal will be to validate both safety and effectiveness of the target compound by identifying the minimal concentration of dissolution agent yielding adequate drainage and minimal tissue injury at long residence times. This model can then be used to identify protocols for the volume of product, dwell times, and lavage modalities yielding the best in vivo dissolution and drainage.

### 3.3 Endoscopic and Percutaneous Application

The presently disclosed foam-based chemical dissolution formulation will be used with, and sold alongside, an endoscopic or percutaneous delivery kit. Approximately 70,000 patients annually require endoscopic interventions for debridement, accounting for around 200,000 procedures annually in the United States alone. Mechanical debriders, such as EndoRotor®, or LAMS are currently used in WON to facilitate repeat endoscopic necrosectomies. These tools, however, are exorbitantly expensive and often require multiple endoscopic procedures thereby adding to costs. The presently disclosed subject matter ensures complete dissolution of debris in the WON in a single endoscopy session, resulting in significant cost savings, expedited patient recovery, and reduced periprocedural complications.

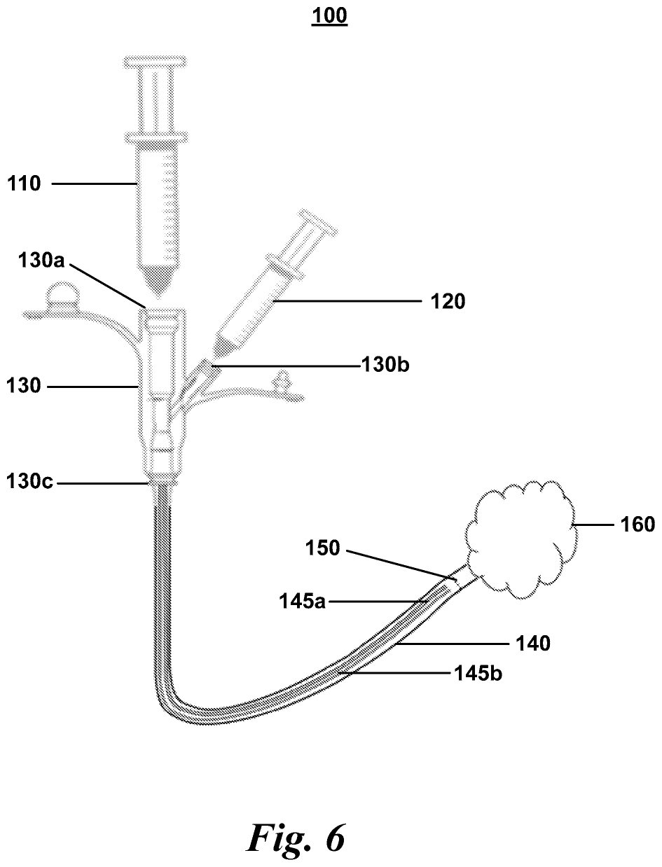

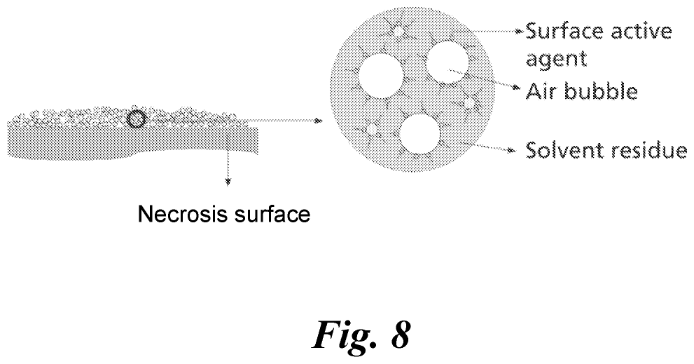

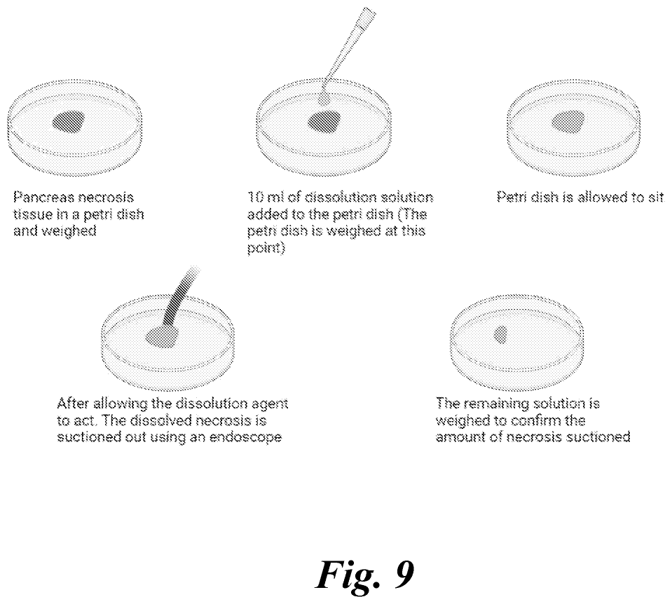

As provided in more detail herein below, an endoscopic delivery system would be comprised of a dual-lumen catheter system that will deliver the dissolution formulation and a propellant to the distal tip of the endoscope and facilitate mixing of the compounds via a foam proportioning system (FIG. 6). The proximal portion of the catheter will include a Y-shaped intake device with a port for injection of the dissolution compound through a pre-loaded syringe. A second intake port will connect to a propellant including compressed air or carbon dioxide source in the procedure suite. The middle portion of the catheter will be comprised of a long flexible catheter containing two lumens connected to the aforementioned intake ports and will be inserted into the instrument port of a standard endoscope. The distal portion of the catheter will contain a dispersion screen to allow for dissolution compound to mix with the propellant to form a foam (FIG. 8). Foam will be propelled through the outflow port at the distal tip of the catheter and into the target area. Directional flow of the foam will be controlled via the endoscope's angulation controls.

Referring now to FIG. 6 is an example of an endoscopic/percutaneous foam delivery catheter comprising a dual-lumen catheter with a distal foam proportioning system. Referring once again to FIG. 6 is endoscopic/percutaneous foam delivery catheter 100. Endoscopic/percutaneous foam delivery catheter 100 comprises a first syringe 110 comprising the presently disclosed dissolution composition and a second syringe 120 comprising a propellent, e.g., compressed air and/or CO₂. First syringe 110 and second syringe 120 are adapted to be in fluid communication with Y-connector 130. Y-connector 130 further comprises a first intake port 130a, which is in fluid communication with first syringe 110, and a second intake port 130b, which is in fluid communication with second syringe 120. First syringe 110 and second syringe 120 can represent any source of the presently disclosed dissolution composition and propellent, respectively. Y-connector 130 is in fluid communication with dual-lumen catheter 140 via output port 130c. Dual-lumen catheter 140 comprises two lumens, propellent lumen 145a and dissolution composition lumen 145b. In some embodiments, propellent lumen 145a is has a larger diameter than dissolution composition lumen 145b. Dual-lumen catheter 140 further comprises dispersion screen 150. In operation, endoscopic/percutaneous foam delivery catheter 100 produces foam 160.

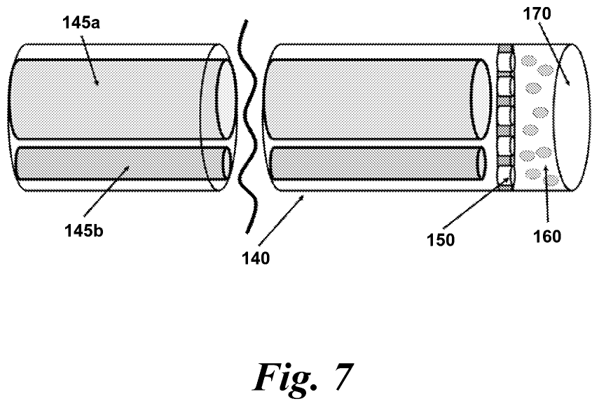

Referring now to FIG. 7, is an expanded view of a portion of endoscopic/percutaneous foam delivery catheter 100. Endoscopic/percutaneous foam delivery catheter 100 comprises propellant lumen 145a, dissolution composition lumen 145b, which are encased in dual-lumen catheter 140. Endoscopic/percutaneous foam delivery catheter 100 further comprises dispersion screen 150, through which aerated foam 160 is generated. Endoscopic/percutaneous foam delivery catheter 100 further comprises dissolution foam outflow port 170.

A variant of this delivery system would be optimized for percutaneous delivery using the same dual lumen design and dispersion screen with a wider range of catheter lengths and diameters.

### 3.4 Applications in Non-Pancreatic Fluid Collections

Beyond WON, the presently disclosed formulation may have applications in necrotic collections or abscesses containing solid debris elsewhere in the human body, including applications in orthopedic applications, including complicated septic arthritis, osteomyelitis, and osteonecrosis; pulmonary applications, including loculated pleural effusions, refractory empyema, necrotizing tumors, and organized hemothorax; dental applications, including periodontal abscesses, endodontic abscesses, osteonecrosis of the jaw, and deep tissue infections of the head and neck; and applications related to non-pancreatic intra-abdominal organs, including refractory hepatic abscesses, necrotizing solid tumors, post-surgical abscesses, complicated appendicitis, complicated diverticulitis, tubo-ovarian abscesses, and others. This product may be used in de-clogging tubes that have been clogged due to bio-film, debris such as gastric feeding tubes. An estimated 19,600 patients in the U.S. each year develop complicated pulmonary infections (Grijalva et al., 2011) that might benefit from chemical tissue debridement; an additional 16,000 patients may benefit in orthopedic applications (Singh et al., 2018), while 126,000 patients may benefit in periodontal and endodontal applications (Eke et al., 2018).

### Example 4

### Representative Foam Formulations

### 4.1 Definition of Foams

In the United States Pharmacopeia 32 (General Chapters: 1151), a foam aerosol is defined as an emulsion containing one or more active ingredients, surfactants, aqueous or non-aqueous liquids, and the propellants. In the European Pharmacopoeia (Monograph: 1105), the definition of a medicated foam is ‘a preparation consisting of large volumes of gas dispersed in a liquid generally containing one or more active substances, a surfactant ensuring their formation and various other excipients.’ Zhao et al., 2010a; Zhao et al., 2010b.

### 4.2 Representative Methods of Foam Preparation

Foams can be prepared by five most commonly used methods: (1) mechanical agitation of a solution, (2) injecting a stream of gas into a liquid, (3) injecting a stream of liquid into a liquid, (4) co-injecting gas and liquid into a chamber, and (5) suddenly reducing the pressure of a solution (emulsion or suspension). Zhao et al., 2010b; Pilpel, 1985.

The stability of product and foam should be evaluated prior to dose application (i.e., inside the canister) and post-dose application, respectively. Gennari et al., 2019. A schematic of foam application is provided in FIG. 8.

TABLE 6
Representative Excipients for Foam Formulation

| | |
|---|---|
| Solvent | distilled water, ethanol, isopropanol, glycerin, propylene glycol, |
| | dimethyl isosorbide, DMSO |
| Foaming agent | cetyl alcohol, cetyl stearyl alcohol, sodium docecyl sulfate, |
| | sodium oleate, sodium stearate, stearic acid, polysorbate 20 |
| Foam stabiliser | xanthan gum, guar gum, hydroxyethyl cellulose, hydroxypropyl |
| | cellulose, hydroxypropyl methylcellulose, methyl cellulose, |
| | agar-agar, alginates, sodium lauryl sulfate, lauryl acid, palmitic |
| | acid, stearic acid, coconut oil, tragacanth gum, gelatin, glycerin |
| Hydrophobic | mineral oils, plant oils, esters (e.g., isopropyl myristate), |
| component, emollient | essential oils, petrolatum, omega-3 polyunsaturated oil, silicon |
| | oil |
| Absorption promoter | ethanol, fatty acids, fatty alcohols |
| Foam breaker | alkyl polysiloxanes, oils, alcohols, fatty alcohols, acetone |
| Antifoaming agent | silicone oil, glycerides, polyamide |

TABLE 8
Foam formulation - a short review

| | Key excipients, concentration | | | | | | |
|---|---|---|---|---|---|---|---|
| | | | | Viscosity- | | | |
| | | | | modifying | | | |
| | | | | agents/Foam | | | |
| | | | Solvents, | stabiliser | Foaming | | |
| Formulation | Propellant | Surfactant | cosolvents | [0.05-2%] | agent | Miscellaneous | Ref. |
| Dermal foam - | — | Labrasol ALF/ | Purified water | Xanthan gum, | — | Verstatil PC, | Falusi, 2022 |
| water based | | Kolliphor RH40 | | Hydroxyethyl- | | Phenoxyethanol | |
| | | | | cellulose (HEC), | | (preservative) | |
| | | | | hyaluronic acid, | | | |
| Topical | propane, butane and | Docosanol, | soybean oil, | myristyl alcohol, | cetostearyl | — | Hazot, 2017 |
| oleaginous foam | isobutane | cyclomethicone | light mineral | coconut oil, | alcohol, | | |
| | | | oil, | beeswax, stearyl | stearic acid | | |
| | | | hydrogenated | alcohol and | | | |
| | | | castor oil | stearic acid | | | |
| Novel topical | tetrafluoroethane | Poloxamer 188, | Water | METHOCEl ™ | — | — | Zhou, 2009 |
| emulsion-based | (HFA134a) and | pluronic F127, | | E4M | | | |
| foams | heptafluoropropane | L62D or L31 | | | | | |
| | (HFA 227) | | | | | | |
| FMX101 4% | Nitrogen Propellant | Cyclomethicone | Hydrogenated | fatty acid or fatty | Cetostearyl | — | Kircik, 2020 |
| Minocycline | (AP-70): Butane, | Docosanol | castor oil, | alcohol. | alcohol, | | |
| Topical Foam | Isobutane, Propane | | Soybean oil, | Myristyl alcohol, | Stearic acid | | |
| | | | Coconut oil, | White wax | | | |
| | | | Light mineral | (beeswax) | | | |
| | | | oil | Stearyl alcohol, | | | |

### Example 5

### Pancreas Necrosis Dissolution Experiments

Pancreas necrosis was obtained from humans during endoscopy. Similar pancreas necrosis was generated in pigs. Experiments were conducted to confirm pancreas necrosis in humans and pigs are similar. Referring now to FIG. 9 are studies to test the efficacy of formulations being prepared to dissolve pancreas necrosis.

### Example 6

### Foam Generator for Endoscopy

The presently disclosed foam generator device aims to increase the effectiveness of direct endoscopic necrosectomy and easily integrate with existing procedures. Requirements for the device include, but are not limited to: generates foam at the catheter/endoscope tip; allows user precise, controlled foam release; fills necrotic debris area cavity (5 cm²-20 cm²); allows greater than about 50% field of view for camera; device has an outer diameter less than about 16 mm; and is compatible with conventional endoscope geometry.

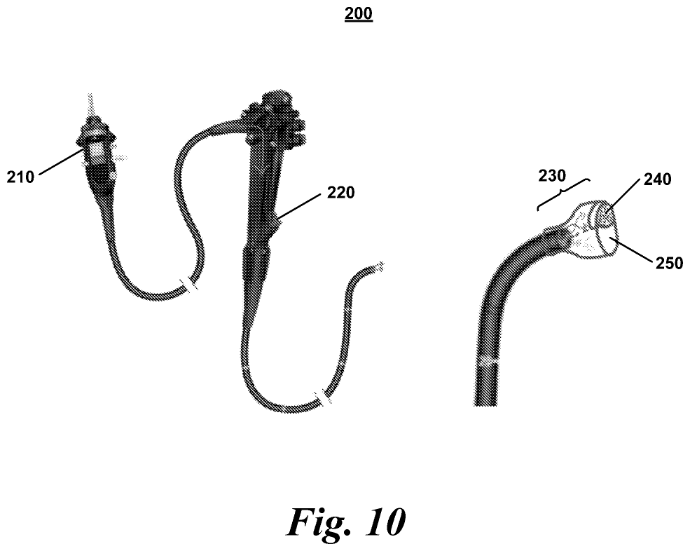

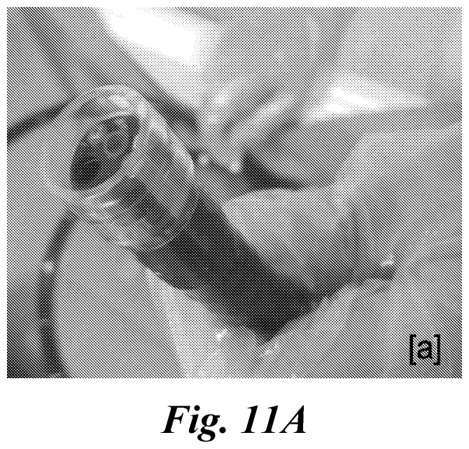

Referring now to FIG. 10 is a representative foam generation endoscopic cap assembly 200. Foam generation endoscopic cap assembly 200 comprises an air insufflation port 210 and a tool port 220 through which the presently disclosed dissolution composition can be injected. Foam generation endoscopic cap assembly 200 comprises foam generation cap 230, which comprises foam generation chamber 240 and hollow cavity 250 for camera view.

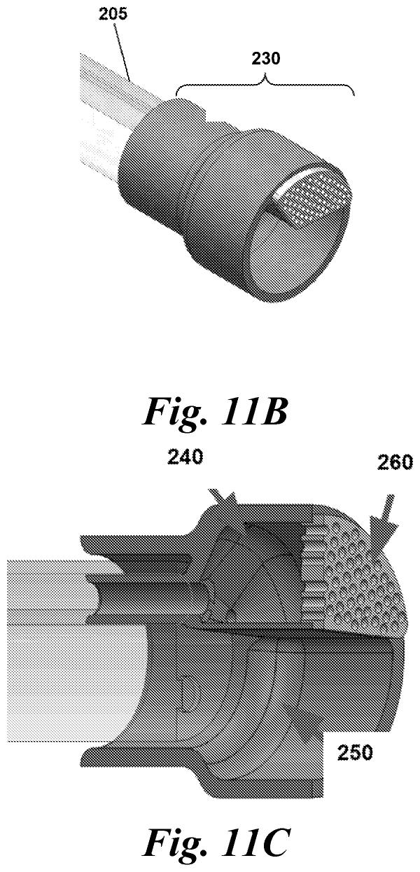

Referring now to FIG. 11A-FIG. 11E the presently disclosed foam generation cap 230 slides onto an endoscope tip and employs a tool port 220 and air insufflation port 210 (FIG. 11E). A dissolution composition is injected through the tool port 220 and the air insufflator port 210 is connected. As provided herein above, foam generation cap 230 comprises foam generation chamber 240 and hollow cavity 250 for camera view (FIG. 11C and FIG. 11E).

More particularly, referring once again to FIG. 11A-FIG. 11E, FIG. 11A is a conventional endoscopic cap known in the art. FIG. 11B is a conventional endoscope distal tip 205 comprising foam generation endoscopic cap 230. FIG. 11C is a section view of foam generation cap 230 comprising foam generation chamber 240, dispersion screen 260, and camera view cavity 250. FIG. 11D is a bottom section view of foam generation cap 230 illustrating flow of the presently disclosed dissolution composition and air/CO₂ into the cap 230 and foam 270 out of foam generation cap 230. FIG. 11E is a side section view of foam generation cap 230 showing a tool port plug 280 and an endoscope camera 290.

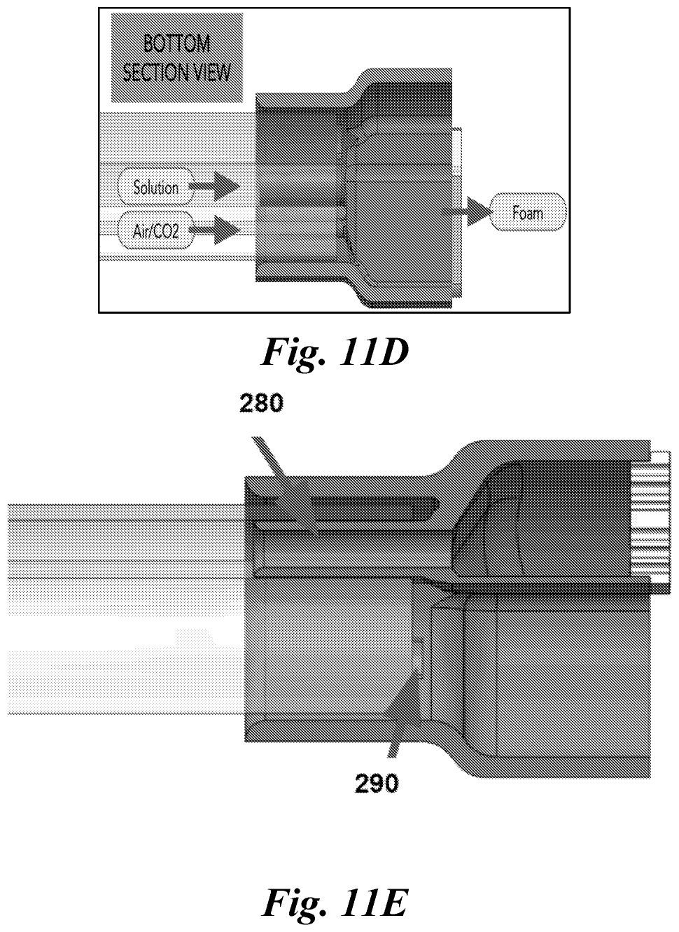

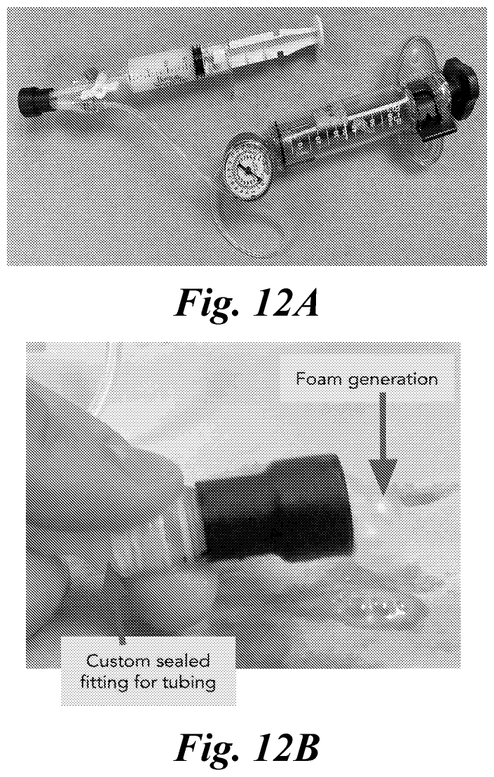

Referring now to FIG. 12A and FIG. 12B, the foam generation endoscopic cap was 3D-printed (PLA) at 1× scale and then fitted onto the endoscope. For initial testing, two syringes and their respective tubes were connected to a 1.2× scale cap to provide the solution and air. Through foam generation chamber inner mesh and dispersion screen adjustments, the cap was able to produce low-stiffness foam (FIG. 12B).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
https://www.pancreapedia.org/reviews/endoscopic-treatment-of-infected-necrosis.
Peery A F, Crockett S D, Murphy C C, Lund J L, Dellon E S, Williams J L, et al. Burden and Cost of Gastrointestinal, Liver, and Pancreatic Diseases in the United States: Update 2018. Gastroenterology. 2019;156(1):254-72.e11. Epub 20181010. doi: 10.1053/j.gastro.2018.08.063. PubMed PMID: 30315778; PubMed Central PMCID: PMCPMC6689327.
Yadav D, Lowenfels A B. The epidemiology of pancreatitis and pancreatic cancer. Gastroenterology. 2013; 144(6):1252-61. Epub 2013 Apr. 30. doi: 10.1053/j.gastro.2013.01.068. PubMed PMID: 23622135; PubMed Central PMCID: PMCPMC3662544.
Banks P A, Bollen T L, Dervenis C, Gooszen H G, Johnson C D, Sarr M G, et al. Classification of acute pancreatitis—2012: revision of the Atlanta classification and definitions by international consensus. Gut. 2013;62(1):102-11. Epub 20121025. doi: 10.1136/gutjnl-2012-302779. PubMed PMID: 23100216.
Baron T H, DiMaio C J, Wang A Y, Morgan K A. American Gastroenterological Association Clinical Practice Update: Management of Pancreatic Necrosis. Gastroenterology. 2020;158(1):67-75.el. Epub 20190831. doi: 10.1053/j.gastro.2019.07.064. PubMed PMID: 31479658.
van Brunschot S, van Grinsven J, van Santvoort H C, Bakker O J, Besselink M G, Boermeester M A, et al. Endoscopic or surgical step-up approach for infected necrotising pancreatitis: a multicentre randomised trial. Lancet. 2018;391(10115):51-8. Epub 20171103. doi: 10.1016/S0140-6736(17)32404-2. PubMed PMID: 29108721.
Chen Y I, Barkun A N, Adam V, Bai G, Singh V K, Bukhari M, et al. Cost-effectiveness analysis comparing lumen-apposing metal stents with plastic stents in the management of pancreatic walled-off necrosis. Gastrointest Endosc. 2018;88(2):267-76.e1. Epub 20180401. doi: 10.1016/j.gie.2018.03.021. PubMed PMID: 29614262.
Abu Dayyeh B K, Mukewar S, Majumder S, Zaghlol R, Vargas Valls E J, Bazerbachi F, et al. Large-caliber metal stents versus plastic stents for the management of pancreatic walled-off necrosis. Gastrointest Endosc. 2018;87(1):141-9. Epub 20170503. doi: 10.1016/j.gie.2017.04.032. PubMed PMID: 28478030.
Gornals J B, Consiglieri C F, Busquets J, Salord S, de-la-Hera M, Secanella L, et al. Endoscopic necrosectomy of walled-off pancreatic necrosis using a lumen-apposing metal stent and irrigation technique. Surg Endosc. 2016;30(6):2592-602. Epub 2015 Sep. 4. doi: 10.1007/s00464-015-4505-2. PubMed PMID: 26335077.
Voermans R P, Besselink M G, Fockens P. Endoscopic management of walled-off pancreatic necrosis. J Hepatobiliary Pancreat Sci. 2015;22(1):20-6. Epub 2014 Oct. 28. doi: 10.1002/jhbp.180. PubMed PMID: 25345777.
Siddiqui A A, Kowalski T E, Loren D E, Khalid A, Soomro A, Mazhar S M, et al. Fully covered self-expanding metal stents versus lumen-apposing fully covered self-expanding metal stent versus plastic stents for endoscopic drainage of pancreatic walled-off necrosis: clinical outcomes and success. Gastrointest Endosc. 2017;85(4):758-65. Epub 20160824. doi: 10.1016/j.gie.2016.08.014. PubMed PMID: 27566053.
Gardner T B, Coelho-Prabhu N, Gordon S R, Gelrud A, Maple J T, Papachristou G I, et al. Direct endoscopic necrosectomy for the treatment of walled-off pancreatic necrosis: results from a multicenter U.S. series. Gastrointest Endosc. 2011;73(4):718-26. Epub 2011 Jan. 18. doi: 10.1016/j.gie.2010.10.053. PubMed PMID: 21237454.
Bang J Y, Arnoletti J P, Holt B A, Sutton B, Hasan M K, Navaneethan U, et al. An Endoscopic Transluminal Approach, Compared With Minimally Invasive Surgery, Reduces Complications and Costs for Patients With Necrotizing Pancreatitis. Gastroenterology. 2019;156(4):1027-40.e3. Epub 20181116. doi: 10.1053/j.gastro.2018.11.031. PubMed PMID: 30452918.
Eke P I, Thornton-Evans G O, Wei L, Borgnakke W S, Dye B A, Genco R J. Periodontitis in US Adults: National Health and Nutrition Examination Survey 2009-2014. J Am Dent Assoc 2018; 149(7): 576-88.e6.
Fugazza A, Sethi A, Trindade A J, Troncone E, Devlin J, Khashab M A, et al. International multicenter comprehensive analysis of adverse events associated with lumen-apposing metal stent placement for pancreatic fluid collection drainage. Gastrointest Endosc. 2020;91(3):574-83. Epub 20191120. doi: 10.1016/j.gie.2019.11.021. PubMed PMID: 31759037.
Cobankara F K, Ozkan H B, Terlemez A. Comparison of organic tissue dissolution capacities of sodium hypochlorite and chlorine dioxide. J Endod. 2010;36(2):272-4. Epub 2010 Feb. 2. doi: 10.1016/j.joen.2009.10.027. PubMed PMID: 20113788.
Stojicic S, Zivkovic S, Qian W, Zhang H, Haapasalo M. Tissue dissolution by sodium hypochlorite: effect of concentration, temperature, agitation, and surfactant. J Endod. 2010;36(9):1558-62. doi: 10.1016/j.joen.2010.06.021. PubMed PMID: 20728727.
Gupta R, Shenvi S D, Nada R, Rana S S, Khullar M, Kang M, et al. Streptokinase may play role in expanding non-operative management of infected walled off pancreatic necrosis: preliminary results. Pancreatology. 2014;14(5):415-8. Epub 20140808. doi: 10.1016/j.pan.2014.07.012. PubMed PMID: 25154047.
Bhargava V, Gupta R, Vaswani P, Jha B, Rana S S, Gorsi U, et al. Streptokinase irrigation through a percutaneous catheter helps decrease the need for necrosectomy and reduces mortality in necrotizing pancreatitis as part of a step-up approach. Surgery. 2021. Epub 20210611. doi: 10.1016/j.surg.2021.05.028. PubMed PMID: 34127302.
Parra V, Kedia P, Zerbo S, Sharaiha R Z, Kahaleh M. Drainage of infected pancreatic necrosis by using 2 lumen-apposing metal stents, a nasocystic drain, and hydrogen peroxide. Gastrointest Endosc. 2015;81(5):1261. Epub 20141031. doi: 10.1016/j.gie.2014.08.009. PubMed PMID: 25442087.
Grijalva C G, Zhu Y, Nuorti J P, Griffin M R. Emergence of parapneumonic empyema in the USA. Thorax 2011; 66(8): 663-8.
Günay S, Paköz B, Çekiç C, Çamyar H, Alper E, Yüksel E S, et al. Evaluation of hydrogen peroxide-assisted endoscopic ultrasonography-guided necrosectomy in walled-off pancreatic necrosis: A single-center experience. Medicine (Baltimore). 2021;100(3):e23175. doi: 10.1097/MD.0000000000023175. PubMed PMID: 33545925; PubMed Central PMCID: PMCPMC7837928.
Maharshi S, Sharma S S, Ratra S, Sapra B, Sharma D. Management of walled-off necrosis with nasocystic irrigation with hydrogen peroxide versus biflanged metal stent: randomized controlled trial. Endosc Int Open. 2021;9(7):E1108-E15. Epub 20210621. doi: 10.1055/a-1480-7115. PubMed PMID: 34222637; PubMed Central PMCID: PMCPMC8216781.
Messallam A A, Adler D G, Shah R J, Nieto J M, Moran R, Elmunzer B J, et al. Direct Endoscopic Necrosectomy With and Without Hydrogen Peroxide for Walled-off Pancreatic Necrosis: A Multicenter Comparative Study. Am J Gastroenterol. 2021;116(4):700-9. doi: 10.14309/ajg.0000000000000987. PubMed PMID: 33982939.
Abdelhafez M, Elnegouly M, Hasab Allah M S, Elshazli M, Mikhail H M, Yosry A. Transluminal retroperitoneal endoscopic necrosectomy with the use of hydrogen peroxide and without external irrigation: a novel approach for the treatment of walled-off pancreatic necrosis. Surg Endosc. 2013;27(10):3911-20. Epub 20130413. doi: 10.1007/s00464-013-2948-x. PubMed PMID: 23584819.
Othman M O, Elhanafi S, Saadi M, Yu C, Davis B R. Extended Cystogastrostomy with Hydrogen Peroxide Irrigation Facilitates Endoscopic Pancreatic Necrosectomy. Diagn Ther Endosc. 2017;2017:7145803. Epub 20170905. doi: 10.1155/2017/7145803. PubMed PMID: 29056844; PubMed Central PMCID: PMCPMC5605784.
de Almeida L H, Leonardo N G, Gomes A P, Souza E M, Pappen F G. Influence of EDTA and dentine in tissue dissolution ability of sodium hypochlorite. Aust Endod J. 2015;41(1):7-11. Epub 2013 Nov. 1. doi: 10.1111/aej.12044. PubMed PMID: 24171764.
Senia E S, Marshall F J, Rosen S. The solvent action of sodium hypochlorite on pulp tissue of extracted teeth. Oral Surg Oral Med Oral Pathol. 1971;31(1):96-103. doi: 10.1016/0030-4220(71)90040-5. PubMed PMID: 5275511.
Shih M, Marshall F J, Rosen S. The bactericidal efficiency of sodium hypochlorite as an endodontic irrigant. Oral Surg Oral Med Oral Pathol. 1970;29(4):613-9. doi: 10.1016/0030-4220(70)90473-1. PubMed PMID: 5264905.
Taylor H D, Austin J H. THE SOLVENT ACTION OF ANTISEPTICS ON NECROTIC TISSUE. J Exp Med. 1918;27(1):155-64. doi: 10.1084/jem.27.1.155. PubMed PMID: 19868192; PubMed Central PMCID: PMCPMC2125954.
Svec T A, Harrison J W. Chemomechanical removal of pulpal and dentinal debris with sodium hypochlorite and hydrogen peroxide vs normal saline solution. J Endod. 1977;3(2):49-53. doi: 10.1016/S0099-2399(77)80015-0. PubMed PMID: 264933.
Cunningham W T, Balekjian A Y. Effect of temperature on collagen-dissolving ability of sodium hypochlorite endodontic irrigant. Oral Surg Oral Med Oral Pathol. 1980;49(2):175-7. doi: 10.1016/0030-4220(80)90313-8. PubMed PMID: 6928291.
Cullen J K, Wealleans J A, Kirkpatrick T C, Yaccino J M. The effect of 8.25% sodium hypochlorite on dental pulp dissolution and dentin flexural strength and modulus. J Endod. 2015;41(6):920-4. Epub 2015 Mar. 21. doi: 10.1016/j.joen.2015.01.028. PubMed PMID: 25791075.
Thé S D, Maltha J C, Plasschaert A J. Reactions of guinea pig subcutaneous connective tissue following exposure to sodium hypochlorite. Oral Surg Oral Med Oral Pathol. 1980;49(5):460-6. doi: 10.1016/0030-4220(80)90292-3. PubMed PMID: 6929469.
Moorer W R, Wesselink P R. Factors promoting the tissue dissolving capability of sodium hypochlorite. Int Endod J. 1982;15(4):187-96. doi: 10.1111/j.1365-2591.1982.tb01277.x. PubMed PMID: 6964523.
Singh J A, Yu S. Septic Arthritis in Emergency Departments in the US: A National Study of Health Care Utilization and Time Trends. Arthritis Care Res (Hoboken) 2018; 70(2): 320-6.
Sukovatykh B S, Blinkov I I, Eshtokin S A, Frolova O G. [Experimental-clinical grounds of the application of immobilized forms of sodium hypochlorite in treatment of diffuse peritonitis]. Vestn Khir Im I Grek. 2008;167(6):44-7. PubMed PMID: 19241815.
Sukovatykh B S, Blinkov I I, Eshotkin S A, Frolova O G. [Immobilized forms of sodium hypochlorite in carboxymetilcellulose gel used in treatment of disseminated peritonitis]. Khirurgiia (Mosk). 2009(11):14-7. PubMed PMID: 20032938.
Sukovatykh B S, Blinkov I I, Bukreeva A E, Eshtokin S A, Ivanov P A, Zhukovskiĭ V A. [The application of immobilized forms of sodium hypochlorite in complex treatment of diffuse purulent peritonitis]. Vestn Khir Im I Grek. 2011;170(6):32-6. PubMed PMID: 22416404.
Sukovatykh B S, Blinkov I I, Ivanov P A, Miliaev E M. [The videoendoscopic sanations of the abdominal cavity by the spread peritonitis]. Khirurgiia (Mosk). 2012(5):18-23. PubMed PMID: 22810530.
Wang Y-N, Brayman A, Chan K, Richmond K, Monsky W, Matula T. A porcine bimicrobial abscess model for testing interventional treatments. 2020.
Zhao, Y., Jones, S. A., and Brown, M. B., “Dynamic foams in topical drug delivery,” J. Pharm. Pharmacol., vol. 62, no. 6, pp. 678-684, 2010a, doi: 10.1211/jpp.62.06.0003.
Zhao, Y., Brown, M. B., and Jones, S. A., “Pharmaceutical foams: are they the answer to the dilemma of topical nanoparticles?,” Nanomedicine Nanotechnology, Biol. Med., vol. 6, no. 2, pp. 227-236, 2010b, doi: 10.1016/j.nano.2009.08.002.
N. Pilpel, N., “Foams in pharmacy,” Endeavour, vol. 9, no. 2, pp. 87-91, 1985.
Gennari, C. G. M., Selmin, F., Minghetti, P., and Cilurzo, F., “Medicated Foams and Film Forming Dosage Forms as Tools to Improve the Thermodynamic Activity of Drugs to be Administered Through the Skin,” Curr. Drug Deliv., vol. 16, no. 5, pp. 461-471, 2019, doi: 10.2174/1567201816666190118124439.
Falusi, F., et al., “Investigation of the effect of polymers on dermal foam properties using the QbD approach,” Eur. J. Pharm. Sci., vol. 173, no. November 2021, 2022, doi: 10.1016/j.ejps.2022.106160.
Hazot, Y., Malinov, T., Gazal, E., Keynan, R., Margulis, L., and Tamarkin, D., “Topical Oleaginous Minocycline Foam: Efficacious Delivery into Skin Layers,” J. Anal. Pharm. Res., vol. 4, no. 5, pp. 1-4, 2017, doi: 10.15406/japlr.2017.04.00117.
Zhao, Y., Brown, M. B., and Jones, S. A., “Engineering novel topical foams using hydrofluroalkane emulsions stabilised with pluronic surfactants,” Eur. J. Pharm. Sci., vol. 37, no. 3-4, pp. 370-377, 2009, doi: 10.1016/j.ejps.2009.03.007.
Kircik, L., et al., “Formulation and Profile of FMX101 4% Minocycline Topical Foam for the Treatment of Acne Vulgaris,” J. Clin. Aesthet. Dermatol., vol. 13, no. 4, pp. 14-21, 2020.

Although the foregoing subject matter has been described in some detail by way of illustration and example for purposes of clarity of understanding, it will be understood by those skilled in the art that certain changes and modifications can be practiced within the scope of the appended claims.

1. An endoscopic foam delivery catheter comprising:
a first syringe comprising a dissolution solution and a second syringe comprising a propellent, wherein the first syringe and the second syringe are adapted to be in fluid communication a Y-connector,
wherein the Y-connector comprises a first intake port in fluid communication with the first syringe, and a second intake port in fluid communication with the second syringe,
wherein the Y-connector is in fluid communication with a dual-lumen catheter via an output port,
wherein the dual-lumen catheter comprises a propellent lumen and dissolution solution lumen, a dispersion screen; a dissolution foam outflow port at a distal end thereof.

2. The endoscopic foam delivery catheter of claim 1, further comprising a foam generation cap comprising a foam generation chamber, a dispersion screen, and a hollow cavity adapted for camera view.

3. The endoscopic foam delivery catheter of claim 2, wherein the foam generation cap further comprises one or more of a tool port plug and an endoscope camera.

4. (canceled)

5. The endoscopic foam delivery catheter of claim 1, wherein the dissolution solution comprises between about 0.5% to about 5% hypochlorite and one or more of a gel formulation, a foam formulation, and combinations thereof.

6. The endoscopic foam delivery catheter of claim 5, wherein the dissolution solution further comprises one or more of:
(a) one or more dissolution compounds selected from chlorine dioxide, hydrogen peroxide, eusol (chlorinated lime in boric acid), ethylenediaminetetraacetic acid (EDTA), formocresol (formaldehyde, cresol, glycerin), glutaraldehyde, one or more proteolytic enzymes, peracetic acid, chlorhexidine, Ca hypochlorite, and papain gel;
(b) an excipient selected from glyceryl monooleate, lecithin, oleic acid, a dibutyl sebacate salt, sodium chloride, one or more preservatives, glycerin, chlorhexidine, dimethyl sulfoxide, glyceryl behenate, sodium stearate, glyceryl palmitostearate, olive oil, and sucrose stearate.

7. The endoscopic foam delivery catheter of claim 6, wherein the one or more proteolytic enzymes include a collagenase.

8. The endoscopic foam delivery catheter of claim 5, wherein the gel formulation comprises one or more components selected from an alginic acid, a cellulosic-based compound, a lectin-like cytoadhesive, gellan gum, polycarbophil, carbomer, chitosan, xanthan gum, cyclomethicone, a poloxamer, polyethylene oxide, carrageenan, carboxymethylcellulose, ammonium alginate, sodium alginate, potassium alginate, calcium alginate, poly(methyl vinyl ether/maleic anhydride) ethyl cellulose, propylene glycol, sodium hyaluronate, agar, dextran hydroxypropyl cellulose, croscarmellose sodium, dimethyl-beta-cyclodextrin, povidone, methylcellulose, potassium alginate, pectin, an aliphatic polyester, ceresin, a polyoxyethylene alkyl ester, ceratonia, gelatin, propylene glycol alginate, polyvinyl alcohol, hydroxyethyl methyl cellulose polyoxyl hydroxystearate, and guar gum.

9. (canceled)

10. The endoscopic foam delivery catheter of claim 5, wherein the foam formulation comprises one or more of a pharmaceutically acceptable vehicle, a surfactant, a propellant, a stabilizing polymer, a buffer, a solvent, a foaming agent, a foam stabilizer, a hydrophobic component and/or emollient, an absorption promoter, a foam breaker, and an antifoaming agent.

11. The endoscopic foam delivery catheter of claim 10, wherein:
(a) the pharmaceutically acceptable vehicle comprises propylene glycol and water;
(b) the surfactant is a polysorbate or a sorbitan ester;
(c) the propellant is selected from isobutane, propane, air, carbon dioxide, and combinations thereof;
(d) the stabilizing polymer is a poloxamer; and/or
(e) the buffer is a phosphate or a citrate buffer.

12-16. (canceled)

17. The endoscopic foam delivery catheter of claim 10, wherein:
(a) the solvent is selected from distilled water, ethanol, isopropanol, glycerin, propylene glycol, dimethyl isosorbide, and dimethyl sulfoxide (DMSO);
(b) the foaming agent is selected from cetyl alcohol, cetyl stearyl alcohol, sodium docecyl sulfate, sodium oleate, sodium stearate, stearic acid, and polysorbate 20;
(c) the foam stabilizer is selected from xanthan gum, guar gum, hydroxyethyl cellulose, hydroxypropyl cellulose, hydroxypropyl methylcellulose methyl cellulose, agar-agar, alginates, sodium lauryl sulfate, lauryl acid, palmitic acid, stearic acid, coconut oil, tragacanth gum, gelatin, and glycerin;
(d) the hydrophobic component and/or emollient is selected from a mineral oil, a plant oil, an ester, an essential oils, petrolatum, omega-3 polyunsaturated oil, and silicon oil;
(e) the absorption promoter is selected from ethanol, a fatty acid, and a fatty alcohol;
(f) the foam breaker is selected from an alkyl polysiloxane, an oil, an alcohol, a fatty alcohol, and acetone; and/or
(g) the antifoaming agent is selected from silicone oil, a glyceride, and polyamide.

18-23. (canceled)

24. The endoscopic foam delivery catheter of claim 10, wherein the foam formulation comprises one or more components selected from NaClO, cetyl alcohol, a polysorbate, glycerin, a polyvinylpyrrolidone, a hydroxypropyl methylcellulose (HPMC), and water.

25. The endoscopic foam delivery catheter of claim 24, wherein the foam formulation comprises between about 0.5% to about 5% NaClO, between about 0.1% to about 3.0% cetyl alcohol, between about 0.1% to about 2.5% of a polysorbate, between about 2.0% to about 6.0% glycerin, between about 0.0% to about 3.0% of a polyvinylpyrrolidone, between about 0.0% to about 3.0% of a hydroxypropyl methylcellulose (HPMC), and water.

26. The endoscopic foam delivery catheter of claim 25, wherein the foam formulation comprises about 3.5% NaClO, about 0.5% cetyl alcohol, about 0.5% of a polysorbate, about 5.0% glycerin, between about 0.0% to about 3.0% of a polyvinylpyrrolidone, between about 0.0% to about 0.3% of a hydroxypropyl methylcellulose (HPMC), and water.

27. A kit comprising the endoscopic foam delivery catheter claim 1, wherein the kit optionally comprises one or more accessories for lavaging the one or more pancreatic necrotic collections and/or draining the one or more pancreatic necrotic collections.

28-29. (canceled)

30. A method for treating pancreatic necrosis, the method comprising administering a dissolution solution with the endoscopic foam delivery catheter claim 1 to one or more pancreatic necrotic collections in a subject in need of treatment thereof.

31. The method of claim 30, comprising one or more of:
(a) dissolving one or more solid necrosomes of the one or more pancreatic necrotic collections;
(b) decreasing a viscosity of a liquid collection of the one or more pancreatic necrotic collections;
(c) lavaging the one or more pancreatic necrotic collections; and
(d) draining the one or more pancreatic necrotic collections with a stent or a pigtail drain.

32-34. (canceled)

35. The method of claim 30, wherein a volume of the dissolution solution administered has a range from about 10 mL to about 100 mL.

36. The method of claim 30, comprising administering the dissolution solution over a period of time having a range from about 1 minute to about 10 minutes.

37. The method of claim 30, wherein the treatment is conducted over one treatment session.

38. A dissolution solution comprising between about 0.5% to about 5% hypochlorite and one or more of a gel formulation, a foam formulation, and combinations thereof, wherein:
(a) the dissolution solution comprises one or more dissolution compounds selected from chlorine dioxide, hydrogen peroxide, eusol (chlorinated lime in boric acid), ethylenediaminetetraacetic acid (EDTA), formocresol (formaldehyde, cresol, glycerin), glutaraldehyde, one or more proteolytic enzymes, peracetic acid, chlorhexidine, Ca hypochlorite, and papain gel, wherein the one or more proteolytic enzymes optionally include a collagenase;
(b) the gel formulation comprises one or more components selected from an alginic acid, a cellulosic-based compound, a lectin-like cytoadhesive, gellan gum, polycarbophil, carbomer, chitosan, xanthan gum, cyclomethicone, a poloxamer, polyethylene oxide, carrageenan, carboxymethylcellulose, ammonium alginate, sodium alginate, potassium alginate, calcium alginate, poly(methyl vinyl ether/maleic anhydride) ethyl cellulose, propylene glycol, sodium hyaluronate, agar, dextran hydroxypropyl cellulose, croscarmellose sodium, dimethyl-beta-cyclodextrin, povidone, methylcellulose, potassium alginate, pectin, an aliphatic polyester, ceresin, a polyoxyethylene alkyl ester, ceratonia, gelatin, propylene glycol alginate, polyvinyl alcohol, hydroxyethyl methyl cellulose polyoxyl hydroxystearate, and guar gum;
(c) the dissolution solution further comprises an excipient selected from glyceryl monooleate, lecithin, oleic acid, a dibutyl sebacate salt, sodium chloride, one or more preservatives, glycerin, chlorhexidine, dimethyl sulfoxide, glyceryl behenate, sodium stearate, glyceryl palmitostearate, olive oil, and sucrose stearate;
(d) the foam formulation comprises one or more of a pharmaceutically acceptable vehicle, a surfactant, a propellant, a stabilizing polymer, and a buffer, wherein optionally (i) the pharmaceutically acceptable vehicle comprises propylene glycol and water; (ii) the surfactant is a polysorbate or a sorbitan ester; (iii) the propellant is selected from isobutane, propane, air, carbon dioxide, and combinations thereof; (iv) the stabilizing polymer is a poloxamer; (v) the buffer is a phosphate or a citrate buffer;
(e) the foam formulation comprises one or more components selected from a solvent, a foaming agent, a foam stabilizer, a hydrophobic component and/or emollient, an absorption promoter, a foam breaker, and an antifoaming agent, wherein optionally (i) the solvent is selected from distilled water, ethanol, isopropanol, glycerin, propylene glycol, dimethyl isosorbide, and dimethyl sulfoxide (DMSO); (ii) the foaming agent is selected from cetyl alcohol, cetyl stearyl alcohol, sodium docecyl sulfate, sodium oleate, sodium stearate, stearic acid, and polysorbate 20; (iii) the foam stabilizer is selected from xanthan gum, guar gum, hydroxyethyl cellulose, hydroxypropyl cellulose, hydroxypropyl methylcellulose methyl cellulose, agar-agar, alginates, sodium lauryl sulfate, lauryl acid, palmitic acid, stearic acid, coconut oil, tragacanth gum, gelatin, and glycerin; (iv) the hydrophobic component and/or emollient is selected from a mineral oil, a plant oil, an ester, an essential oils, petrolatum, omega-3 polyunsaturated oil, and silicon oil; (v) the absorption promoter is selected from ethanol, a fatty acid, and a fatty alcohol; (vi) the foam breaker is selected from an alkyl polysiloxane, an oil, an alcohol, a fatty alcohol, and acetone; (vii) the antifoaming agent is selected from silicone oil, a glyceride, and polyamide;
(f) the foam formulation comprises one or more components selected from NaClO, cetyl alcohol, a polysorbate, glycerin, a polyvinylpyrrolidone, a hydroxypropyl methylcellulose (HPMC), and water;
(g) the foam formulation comprises between about 0.5% to about 5% NaClO, between about 0.1% to about 3.0% cetyl alcohol, between about 0.1% to about 2.5% of a polysorbate, between about 2.0% to about 6.0% glycerin, between about 0.0% to about 3.0% of a polyvinylpyrrolidone, between about 0.0% to about 3.0% of a hydroxypropyl methylcellulose (HPMC), and water; and/or
(h) the foam formulation comprises about 3.5% NaClO, about 0.5% cetyl alcohol, about 0.5% of a polysorbate, about 5.0% glycerin, between about 0.0% to about 3.0% of a polyvinylpyrrolidone, between about 0.0% to about 0.3% of a hydroxypropyl methylcellulose (HPMC), and water.

39-59. (canceled)
